(19) United States
(12) Patent Application Publication
(10) Pub. No.: US 2003/0186862 A1
(43) Pub. Date: Oct. 2, 2003

(54) Factor VIIa compositions

(76) Inventor: Gary L. Nelsestuen, St. Paul, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/115,166

(22) Filed: Apr. 2, 2002

(51) Int. Cl. A61K 38/36

(52) U.S. Cl. 514/12

(57) ABSTRACT

Compositions that include a factor VIIa polypeptide and a factor X polypeptide are described. Such compositions are free of thrombogenic levels of active clotting factors, other than factor VIIa, and can be used to increase clot formation in a mammal.

### TECHNICAL FIELD

This invention relates to factor VIIa compositions, and more particularly to factor VIIa compositions containing factor VIIa and factor X.

### BACKGROUND

Bleeding problems arise in hemophilia and other disorders such as cancer and liver disease. Treatment of these problems often involves administering factor VIII or factor IX. Unfortunately, this therapy does not always work. Other treatments include administering prothrombin complex concentrates (PCC) or recombinant human factor VIIa at high doses (e.g., 400 times the level of circulating factor VIIa). PCC is a relatively crude preparation isolated from blood, and contains many vitamin K-dependent proteins, including prothrombin, factor X, factor IX, and factor VII, as well as proteins S, C, and Z. The mechanism by which PCC works is not known, although increasing levels of coagulation proteins in the circulation, introducing active clotting factors, or inducing tissue factor expression on the surface of endothelial cells have been proposed. Certain commercial preparations of PCC are treated in a manner to increase the levels of active enzymes within PCC. The mechanism by which factor VIIa functions also is uncertain. Factor VIIa may directly activate factor X in a tissue factor dependent or independent mechanism. Results from factor VIIa and PCC therapy are inconsistent and treatment frequently fails. See, Lusher et al., (1998) Blood Coagul. Fibrinolysis, 9(2):119-28. As a result, patients may be treated in many different ways before hemostasis can be attained.

### SUMMARY

The invention is based on the discovery that factor VIIa can be simultaneously administered to a mammal with factor X when the amount of factor X is not thrombogenic. As a result, a composition containing factor VIIa and factor X can be formulated and used for treating clotting disorders. Factor VIIa converts factor X to factor Xa, which then activates prothrombin to thrombin, which, in turn, cleaves fibrinogen and causes blood to clot. Since the composition is free of thrombogenic levels of active clotting factors, other than factor VIIa (e.g., any factor Xa polypeptide has been removed or inhibited), risks of thrombogenic complications are reduced in the patient. Thus, compositions of the invention are safer and more effective for treating clotting disorders. The combined administration of factor VIIa and factor X results in a superior coagulation stimulus that can function better than using either reagent alone.

In one aspect, the invention features a composition that includes a factor VIIa polypeptide and a factor X polypeptide, wherein the composition is free of thrombogenic levels of active clotting factors, other than factor VIIa. The composition contains an amount of the factor VIIa polypeptide which, upon administration to a human patient, produces an increase of 0.1 to 5 units of factor VIIa/mL of blood in the patient. The amount of the factor X polypeptide can produce, upon administration to a human patient, an increase of 1 to 10 units of factor X/mL of blood in the patient. The composition further can include a pharmaceutically acceptable carrier. Clot formation can be increased in a patient by administering to the patient an amount of such compositions effective for increasing clot formation. Clotting time of the patient can be monitored.

In another aspect, the invention features a method for increasing clot formation in a patient. The method includes providing a composition that contains a factor VIIa polypeptide and a factor X polypeptide, inhibiting any active clotting factors other than factor VIIa within the composition to form an inhibited composition; and administering to the patient an amount of the inhibited composition effective for increasing clot formation in the patient. The method further can include administering a factor VIIa polypeptide to the patient and/or monitoring clotting time of the patient.

The inhibiting step can include contacting the composition with a sample of the patient's blood for a duration effective to inhibit any active clotting factors, other than factor VIIa, within the composition, wherein the contacting step is performed in the presence of a calcium chelator.

The inhibiting step also can include contacting the composition with amounts of plasma and heparin (e.g., low molecular weight heparin) sufficient to inhibit any active clotting factors, other than factor VIIa, within the composition, wherein the contacting step is performed in the presence of a calcium chelator. The composition can be contacted with an amount of heparin that results, upon administration to the patient, in less than 0.01 unit of heparin per mL of the patient's blood.

The inhibiting step also can include contacting the composition with an amount of a protease inhibiter effective for inhibiting any active clotting factors, other than factor VIIa, within the composition. The protease inhibitor can be a serine protease inhibitor (e.g., antithrombin III) or α-2 macroglobulin. The contacting step further can include contacting the composition with an amount of heparin which, upon administration to the patient, results in less than 0.01 unit per mL of the patient's blood.

The contacting step also can include contacting the composition with an active site inhibitor of a serine protease in the absence of calcium or in the presence of a calcium chelator.

The invention also features a method for increasing clot formation in a patient that include administering to the patient a factor X polypeptide and a factor VIIa polypeptide in amounts effective for increasing clot formation in the patient, wherein the amount of the factor X polypeptide is free of thrombogenic levels of activated clotting factors, and wherein the amount of the factor VIIa produces, upon administration to a human patient, an increase of 0.1 to 5 units of factor VIIa/mL of blood in the patient. The method further can include measuring blood clotting time of the patient. The factor X and factor VIIa polypeptides can be administered sequentially. The factor VIIa and factor X polypeptides can be administered in plasma or in whole bood.

In another aspect, the invention features a composition that includes a factor VIIa polypeptide, a factor X polypeptide, anti-thrombin III, and heparin, wherein the composition is free of thrombogenic levels of active clotting factors, other than factor VIIa.

In yet another aspect, the invention features a composition that includes plasma or blood, a factor VIIa polypeptide, and a factor X polypeptide, wherein the composition is free of thrombogenic levels of active clotting factors, other than factor VIIa, and wherein the amount of the factor VIIa polypeptide produces, upon administration to a human subject, an increase of 0.1 to 5 units of factor VIIa/mL of blood in the human subject.

The invention also features an article of manufacture that includes a first and a second container. The first container includes a factor VIIa polypeptide, wherein the amount of the factor VIIa polypeptide produces, upon administration to a human subject, an increase of 0.1 to 5 units of factor VIIa/mL of blood of the human subject. The second container includes a factor X polypeptide, wherein the factor X polypeptide is free of thrombogenic levels of active clotting factors. The article of manufacture further can include a label indicating that the factor VIIa polypeptide and the factor X polypeptide can be used to increase clot formation in a mammal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1 is a graph depicting the clotting time of blood using different levels of factor VIIa at a constant level of factor X.

FIG. 2 is a graph depicting the clotting time of blood using different levels of added factor X at a constant level of factor VIIa. The three curves show results for no factor VIIa, 12.5 nM factor VIIa, and 50 nM factor VIIa. Zero factor X corresponds to normal factor X levels in the blood (about 60 nM).

### DETAILED DESCRIPTION

In general, the invention features compositions that contain a factor VIIa polypeptide and a factor X polypeptide. Such compositions are free of thrombogenic levels of active clotting factors, other than factor VIIa. Factor X and factor VIIa work synergistically to increase clotting formation in patients. In some embodiments, the composition contains purified factor VIIa and factor X polypeptides and is substantially free of intact red or white blood cells, and other components of blood, such as serum proteins (e.g., albumin). In other embodiments, the composition can include blood or plasma with super endogenous levels of factor VIIa and/or factor X. Using the compositions described herein can be effective for increasing clot formation in mammals, and as a result, can be used for treating patients with hemophilia or other clotting disorders. Compositions of the invention also may be combined with other therapeutic methods in the management of clotting disorders.

Factor VIIa Compositions

Compositions of the invention include a factor VIIa polypeptide and a factor X polypeptide. Factor VIIa is the activated form of factor VII, which is a zymogen. Factor VIIa is generated by proteolytic cleavage at the arginine 152, isoleucine 153 bond of factor VII, and is a serine protease. As used herein, the term “factor VIIa polypeptide” refers to a chain of amino acid residues of any length that has the ability to bind membranes and cofactors, has serine protease activity, and fulfills the role of factor VIIa in a coagulation reaction. Non-limiting examples of suitable factor VIIa polypeptides include native or wild-type factor VIIa (e.g., wild-type human factor VIIa), or modified factor VIIa polypeptides that contain one or more amino acid substitutions, deletions, or insertions relative to wild-type factor VII. Particularly useful modified factor VIIa polypeptides have enhanced membrane binding affinity and/or activity. See, for example, the factor VIIa polypeptides of U.S. Pat. No. 6,017,882 and Shah et al. (1998) Proc. Natl. Acad. Sci. USA 95:4229-4234 (e.g., factor VIIa containing a glutamine at position 10 and a glutamic acid residue at position 32).

Furthermore, suitable factor VIIa polypeptides can be modified such that their circulation half-life is increased. For example, one or more polyethylene glycol (PEG) molecules can be linked to a factor VIIa polypeptide as described in WO 02/02764. Using factor VIIa polypeptides with enhanced membrane-binding affinity, activity, or half-life can reduce the amount of factor VIIa needed in the composition and can decrease the frequency of administration of the composition. As a result, costs associated with treating patients with clotting disorders can be reduced, allowing the therapies to be made more widely available to individuals in need of such pro-coagulation therapies.

Typically, compositions of the invention contain an amount of factor VIIa polypeptide that will, upon a single bolus administration or upon multiple administrations over 4-6 hours or several days to a human patient, produce an increase in 0.1 to 5 whole blood clotting units of factor VIIa/mL of blood or 7.5 to 375 whole blood clotting units per kg body weight of the patient. For example, the composition can contain an amount of factor VIIa sufficient to produce an increase of 0.25 to 5, 0.5 to 5, 1 to 5, 1.5 to 4.5, or 2 to 3 units of factor VIIa/mL of blood in the patient. Factor VIIa levels can be assessed in the patient approximately 30 minutes after injection. The terms “whole blood clotting units” and “units” are used interchangeably throughout the application. One whole blood clotting unit of factor VIIa activity is the amount of factor VIIa that, when added to one mL of normal-response blood (NRB), produces a clotting time of 370 seconds in the Hemochron Jr. Signature Microcoagulation instrument (International Technidyne, Inc.) using the ACT-low range (LR) cuvette and recalcified blood (as described in Example 1). NRB is defined as blood that clots in 370 seconds when factor VIII:C has been removed and 50 nM purified, recombinant factor VIIa has been added. For wild-type factor VIIa, one whole blood clotting unit is approximately 2.5 μg of factor VIIa or 50 nM of factor VIIa in the recalcified blood. For modified factor VIIa polypeptides with enhanced membrane binding or activity, one unit will be less than 2.5 μg. As there are approximately 75 mL of blood per kg body weight, a dose of one unit of factor VIIa per mL in a human corresponds roughly to 75 units of factor VIIa per kg of body weight.

The normal level of factor VIIa in the plasma is 1% of total factor VIIa (10 nM in plasma, 6 nM in whole blood) or about 0.1 nM factor VIIa in plasma (0.06 nM in whole blood) (Morrissey et al. (1993) Blood 81:734-744). Thus, the compositions of the invention produce about 5 to 250 nM factor VIIa in the blood, a level that ranges from about 75 to 4100 times the normal level of factor VIIa in the blood. These levels also are above any dosages that may have been used for combined therapy with factor VIIa and factor X.

As indicated above, compositions of the invention also include a factor X polypeptide. Factor X is a substrate for factor VIIa and helps factor VIIa to decrease clotting time. As used herein, the term “factor X polypeptide” refers to a chain of amino acid residues of any length that can be activated by factor VIIa and has the ability to bind appropriate membranes, and, after activation, fulfills the role of factor Xa in the coagulation cascade. Non-limiting examples of suitable factor X polypeptides include native or wild-type factor X (e.g., wild-type human factor X), or modified factor X polypeptides that contain one or more amino acid substitutions, deletions, or insertions relative to wild-type factor X. Particularly useful modified factor X polypeptides have enhanced membrane binding affinity or activity. See, for example, WO 00/66753 for a description of factor X polypeptides with increased membrane binding affinity and/or activity.

Typically, compositions of the invention contain an amount of a factor X polypeptide that, upon a single bolus administration or upon multiple administrations over several hours or several days to a human patient, produces an increase of 1 to 10 whole blood clotting units of factor X/mL of blood in the patient (260 to 2600 μg factor X/kg of body weight of the patient). For example, the composition may contain an amount of factor X sufficient to produce 1.5 to 10, 2 to 10, 2.5 to 9, 3 to 9, 4 to 8, or 5 to 7 units of factor X/mL of blood in the patient. One unit of factor X is the amount of factor X that decreases clotting time of NRB to the same extent as the addition of a second unit of factor VIIa. More specifically, one unit of factor X is equal to the amount of factor X that, when added to one mL of NRB containing one unit of factor VIIa, will lower clotting time to 0.89× the clotting time without added factor X. Clotting time is measured as set forth above and in Example 1. If one unit of factor VIIa per mL produces a clotting time of approximately 370 seconds, then addition of one unit of factor X will produce a clotting time of approximately 329 seconds. For wild-type factor X, one unit is approximately 3.5 μg or 60 nM factor X per mL of blood in the patient.

Administering one unit of factor X approximately doubles the normal amount of factor X in the blood, which is approximately 60 nM. The amount of factor X in the composition may produce up to 600 nM of factor X in the blood, which is 10 times the normal blood level. This range of amounts for factor X also is greater than factor X amounts that may have been used for combined therapy with factor VIIa. In observing patients that had been receiving PCC for various bleeding episodes, the levels of factor X in the blood can be up to four times higher than normal 20 hours after the last PCC injection. Clotting time of the in vitro test is not improved in such patients, however, until a sufficient amount of factor VIIa is added. Blood from patients treated with PCC showed higher responsiveness to factor VIIa than blood from the same patients before PCC treatment.

Production of Factor VIIa or Factor X Polypeptides

Purified factor VIIa and factor X are commercially available as zymogens or activated factors from, for example, Novo Nordisk (Princeton, N.J.) or Enzyme Research Laboratories (South Bend, Ind.). Alternatively, factor VII and factor X can be purified from plasma or can be produced recombinantly in cell culture or in transgenic animals. Preferably, the transgenic animal or cell culture can carboxylate the glutamic acid residues of the factor VII or factor X polypeptide. To produce such polypeptides by cell culture, a nucleic acid encoding the polypeptide is ligated into a nucleic acid construct such as an expression vector, and host cells are transformed with the expression vector.

In general, nucleic acid constructs include a regulatory sequence operably linked to a nucleic acid sequence that encodes a factor VII or factor X polypeptide. The nucleic acids encoding wild-type factor VII and factor X are known and are available in GenBank (Accession Nos. M13232 and M22613, respectively). Regulatory sequences do not typically encode a gene product, but instead affect the expression of the nucleic acid sequence. As used herein, “operably linked” refers to connection of the regulatory sequences to the nucleic acid sequence in such a way as to permit expression of the nucleic acid sequence. Regulatory elements can include, for example, promoter sequences, enhancer sequences, response elements, or inducible elements.

In eukaryotic host cells, a number of viral-based expression systems can be utilized to express factor VII or factor X. A nucleic acid encoding factor VII or factor X can be cloned into, for example, a baculoviral vector such as pBlueBac (Invitrogen, San Diego, Calif.) and then used to co-transfect insect cells such as Spodoptera frugiperda (Sf9) cells with wild-type DNA from Autographa californica multiply enveloped nuclear polyhedrosis virus (AcMNPV). Recombinant viruses producing the modified vitamin K-dependent polypeptides can be identified by standard methodology. Alternatively, a nucleic acid encoding a vitamin K-dependent polypeptide can be introduced into a SV40, retroviral, or vaccinia based viral vector and used to infect suitable host cells.

Mammalian cell lines that stably express factor VII or factor X can be produced by using expression vectors with the appropriate control elements and a selectable marker. For example, the eukaryotic expression vector pCDNA.3.1+ (Invitrogen, San Diego, Calif.) is suitable for expression of factor VII or factor X in, for example, COS cells, HEK293 cells, or baby hamster kidney cells. Following introduction of the expression vector by electroporation, DEAE dextran, calcium phosphate, liposome-mediated transfection, or other suitable method, stable cell lines can be selected. Alternatively, transiently transfected cell lines are used to produce factor VII or factor X. Factor VII or factor X polypeptides also can be transcribed and translated in vitro using wheat germ extract or rabbit reticulocyte lysate.

Factor VII or factor X polypeptides can be purified from conditioned cell medium by applying the medium to an immunoaffinity column. For example, an antibody having specific binding affinity for factor VII can be used to purify factor VII. Such antibodies can be calcium dependent or independent monoclonal antibodies. Alternatively, concanavalin A (Con A) chromatography and anion-exchange chromatography (e.g., DEAE) can be used in conjunction with affinity chromatography to purify factor VII.

Factor VIIa can be obtained by factor Xa cleavage of factor VII. For example, factor VII and bovine factor Xa (1:100 weight ratio, respectively) can be incubated for 1 hour at 37° C. Factor VIIa also can be obtained by autoactivation. Factor VII can be incubated with soluble tissue factor and phospholipid for 20 minutes at 37° C. Alternatively, factor VIIa can be produced by incubating factor VII and phospholipid for 1 hour at 37° C. Often, factor VIIa is produced during purification steps so that no further activation is necessary.

Methods of Making Factor VIIa Compositions

Compositions of the invention can be prepared by combining a purified factor VIIa polypeptide with a source of factor X polypeptide, such as PCC or a purified factor X polypeptide, such that the composition is free of thrombogenic levels of activated clotting factors, other than factor VIIa. The term “purified” refers to a polypeptide that has been separated from cellular components that naturally accompany it. Typically, the polypeptide is isolated when it is at least 70% (e.g., 80%, 85%, 90%, 95%, or 99%), by weight, free from proteins and naturally-occurring organic molecules that are naturally associated with it. In general, a purified polypeptide will yield a single major band on a non-reducing polyacrylamide gel.

“Thrombogenic levels of activated clotting factors” refers to the combined amount of activated clotting factors, other than factor VIIa, which produces a clotting time less than approximately 400 seconds, in the absence of added factor VIIa, in the clotting assay described herein.

To ensure that compositions of the invention are free of thrombogenic levels of activated clotting factors, the composition can be treated such that any active clotting factors, other than factor VIIa, are inhibited. Alternatively, components of the composition can be treated independently, then mixed together to form a composition. There are various methods to inhibit active clotting factors. Typically, when a composition is treated, the inhibitory reactions are performed in the absence of calcium (e.g., calcium free solutions are used or a calcium chelator such as sodium citrate is present) to prevent inhibition of factor VIIa.

In one method, factor VIIa and a source of factor X can be added to whole blood (e.g., a sample of blood from the patient that the composition will be administered to) and the resulting mixture incubated at room temperature until only non-thrombogenic levels of activated clotting factors, other than factor VIIa, are present. The composition also can be mixed with plasma and a trace amount of heparin (e.g., low molecular weight heparin), then incubated at room temperature until only non-thrombogenic levels of activated clotting factors, other than factor VIIa, are present. Trace amounts of heparin result in less than 0.01 units of heparin per mL of the patient's blood after administration of the composition to the patient. In both of these methods, active clotting factors typically are inhibited after a 30 to 60 minute incubation. Thus, a composition of the invention can include blood or plasma with super endogenous levels of factor VIIa and/or factor X.

In other methods, the composition is combined with a serine protease inhibitor (e.g., human antithrombin III) or other protease inhibitor (e.g., α-2 macroglobulin) in the absence of calcium. Active-site directed inhibitors such as phenylalanylprolylarginine chloromethylketone (PPACK), N-dansyl-glutamyl glycylarginylchloromethylketone (DEGR), and phenylalanyl-phenylalanyl arginylchloromethylketone (FFR), also can be used to inhibit active clotting factors.

If PCC is used as the source of factor X polypeptide, it may be necessary to perform one or more protein purification techniques to enhance the purity of the factor X polypeptide and to reduce the thrombogenic activity of the preparation. One unit of PCC (as defined by the manufacturer, Baxter Hyland Immune, Glendale, Calif.) per mL of blood produces a clotting time of 280 seconds in the clotting assay described herein and would be considered thrombogenic. A single anion exchange column containing DEAE or QEAE resins can be used to remove unknown thrombogenic materials and/or coagulation inhibitor proteins (e.g., protein C, S, or Z) from PCC such that it is no longer thrombogenic.

Pharmaceutical Compositions

Compositions of the invention can be formulated into pharmaceutical compositions by admixture with pharmaceutically acceptable non-toxic excipients or carriers, and used to regulate coagulation in vivo. Generally, the composition can be administered by any suitable route of administration, including orally, transdermally, intravenously, subcutaneously, intramuscularly, intraocularly, intraperitoneally, intrarectally, intravaginally, intranasally, intragastrically, intratracheally, intrapulmonarily, or any combination thereof. Compositions can be prepared for parenteral administration, particularly in the form of liquid solutions or suspensions in aqueous physiological buffer solutions; for oral administration, particularly in the form of tablets or capsules; or for intranasal administration, particularly in the form of powders, nasal drops, or aerosols. Parenteral administration is particularly useful. Compositions for other routes of administration may be prepared as desired using standard methods.

Formulations for parenteral administration may contain as common excipients sterile water or saline, polyalkylene glycols such as polyethylene glycol, vegetable oils, hydrogenated naphthalenes, and the like. In particular, biocompatible, biodegradable lactide polymer, lactide/glycolide copolymer, or polyoxethylene-polyoxypropylene copolymers are examples of excipients for controlling the release of a composition in vivo. Other suitable parenteral delivery systems include ethylene-vinyl acetate copolymer particles, osmotic pumps, implantable infusion systems, and liposomes. Formulations for parenteral administration also may include glycocholate for buccal administration.

Methods of Increasing Clot Formation in Mammals

Compositions of the invention can be administered to patients in need thereof (e.g., hemophilia patients, cancer patients, or patients with liver disease). Alternatively, the individual components of the composition (i.e., factor VIIa and factor X polypeptide) can be administered individually, but within an appropriate time frame (e.g., 60 minutes) to maximize the advantages of simultaneous administration. For example, factor X and factor VIIa polypeptides can be administered sequentially.

A patient's clotting activity can be assessed before administering a composition to determine the patient's overall responsiveness to factor VIIa alone or in combination with factor X. Such an assessment allows the amount of factor VIIa and factor X in the composition to be tailored to the particular patient.

Before administering a composition to a patient, any active clotting factors within the composition, other than factor VIIa, are inhibited or removed as described above. For example, the composition can be contacted with a blood or plasma sample from the patient to which the composition will be administered. Alternatively, in patients undergoing plasmapheresis, the composition can be mixed with donor plasma. In other embodiments, the factor X polypeptide is incubated with a blood or plasma sample as described above to inhibit any active clotting factors, then administered to the patient. Factor VIIa can be subsequently administered to the patient within an appropriate time frame as discussed above.

The dosage of composition required to increase clot formation in the mammal depends on the route of administration, the nature of the composition, the subject's size, weight, surface area, age, and sex, other drugs being concurrently administered, and the judgment of the attending physician. Wide variations in the needed dosage are to be expected in view of the variety of compositions that can be produced (e.g., with wild-type or modified polypeptides), the variety of subjects to which the composition can be administered, and the differing efficacies of various routes of administration. In general, doubling the amount of factor X in the blood (e.g., from 60 nM to 120 nM) doubles the potency of the administered factor VIIa. Patients who have undergone substantial bleeding may benefit from higher dosages of factor X than other patients as factor X levels may be reduced in their circulation. Such patients also may have lowered platelet and blood cell levels, reducing the amount of membrane available to support coagulation reactions, another basis to increase the combined dosage. For example, for a challenging bleed, a large dose can include 1.0 unit of factor VIIa and 4.0 units of factor X per mL of blood. A low dose may include 0.1 unit of factor VIIa plus 4.0 units of factor X per mL of blood or 0.25 units of factor VIIa and 1.0 units of factor X.

The ratio of factor VIIa to factor X in the preparation also can be adjusted based on prior treatments. Since factor VII has a short half-life in the circulation (2-3 hours, Lindley et al., (1994) Clin. Pharmac. Therap., 55:638-648), the initial dosage administered for a bleeding episode may be high in both factor VIIa and X, in order to reach the appropriate blood level.

After a composition is administered to the patient, clotting time can be monitored to evaluate the therapy. It may be desirable to administer additional factor VIIa due to the short half-life of factor VIIa in the circulation. For example, booster injections of factor VIIa can be administered alone. Alternatively, higher levels of factor VIIa can be administered in combination with lower levels of factor X. Both will replace the amounts of the respective protein that are lost from the circulation. In this way, the level of both clotting factors can be maintained at appropriate levels for longer durations.

For chronic management of clotting disorders, the clotting assay described herein can be used to set a range of acceptable dosages for the patient's home therapy since individuals tend to give similar results over time. A patient's blood can be tested in vitro by adding a composition of the invention to a sample of the patient's blood and assessing clotting time. In this way, a specific clotting time can be targeted for all individuals rather than a single dosage for all patients, which is the current practice.

Articles of Manufacture

Compositions described herein can be combined with packaging materials and sold as articles of manufacture or kits. Components and methods for producing articles of manufactures are well known. The articles of manufacture may combine one or more compositions described herein. The article of manufacture also can include two containers, one containing a factor VIIa polypeptide and the other containing a factor X polypeptide. In addition, the articles of manufacture may further include one or more of the following: sterile water, pharmaceutical carriers, buffers, antibodies (e.g., anti-factor VIII:C or anti-factor IX), calcium chelators, calcium containing solutions, and/or other useful reagents for inhibiting active clotting factors, measuring clotting activity, or treating clotting disorders. For example, a kit can include anti-factor VIII:C or anti-factor IX antibodies, citrate or other calcium chelator, and a CaCl₂ solution. A kit also can include factor VIIa and factor X polypeptides, anti-thrombin III, and heparin. A label or instructions describing how factor X and factor VIIa can be used for treatment of clotting disorders (e.g., for increasing clot formation in a hemophiliac) may be included in such kits. The compositions or individual components may be provided in a pre-packaged form in quantities sufficient for single or multiple administrations.

The invention will be further described in the following examples, which do not limit the scope of the invention described in the claims.

### EXAMPLES

The following materials were used unless otherwise indicated. Recombinant factor VIIa (NovoSeven®) was obtained from Novo Nordisk, Princeton, N.J. Purified factor X was obtained from Enzyme Research Laboratories, Inc, South Bend, Ind. PCC (FEIBA) was obtained from Baxter Hyland Immune (Glendale, Calif.). Low molecular weight heparin (LMWH, Lovenox) was obtained from Rhone-Poulenc Rorer Pharmaceuticals, Inc.

### Example 1

In vitro clotting assay: Whole blood was analyzed in the Hemochron Jr. Signature Microcoagulation instrument (International Technidyne, Inc.) using the ACT-low range (LR) cuvette. See also Nelsestuen et al. (2001) Abstract P1397 from the XVIII Congress of the International Society of Thrombosis and Haemostasis. The ACT-LR cuvette contains celite to active the intrinsic coagulation cascade and no added phospholipid. Celite is not necessary to perform the assay. With this instrument and cuvette, normal blood coagulates in 160±20 seconds, blood from severe hemophiliacs coagulates in >400 seconds, and blood from patients with 1% factor VIII or IX coagulates in 357 seconds.

To perform the assay, blood was drawn from an individual and nine volumes of the blood mixed with 1 volume of 0.1 M sodium citrate (or 1 volume of another calcium chelator). The samples were stored in 14 mL plastic conical tip tubes with screw top caps, each containing about 2 mL of blood. Blood from individuals with severe hemophilia can be analyzed directly. When using blood from a normal individual, factor VIII:C was removed. Affinity-purified anti-human factor VIII:C antibodies were added to the chelated blood in an amount sufficient to block all detectable factor VIII:C. This amount was estimated by determining if clotting time of the blood increased to 400 seconds. Typically, 6-8 μg of anti-human factor VIII:C (Affinity Biologicals, Inc., Hamilton, Ontario) were added per mL of blood. Removing the available factor VIII:C renders the contact pathway of coagulation ineffective, and the observed clotting time in the assay is due to other clotting reactions, such as factor X activation by factor VIIa.

After incubating the blood and anti-human factor VIII:C antibody for about an hour at room temperature, the clotting assay was performed. The cells in the tube were suspended by tipping the tube about five or six times. The blood was re-calcified by mixing 0.1 mL of blood with 2.4 μL of 0.4 M CaCl₂ in a small plastic tube. Factor VIIa (≦2 μL, 50 nM) was added to the tube and mixed, then transferred to an LR-cuvette. Clotting time was measured by the Hemochron Jr. instrument.

### Example 2

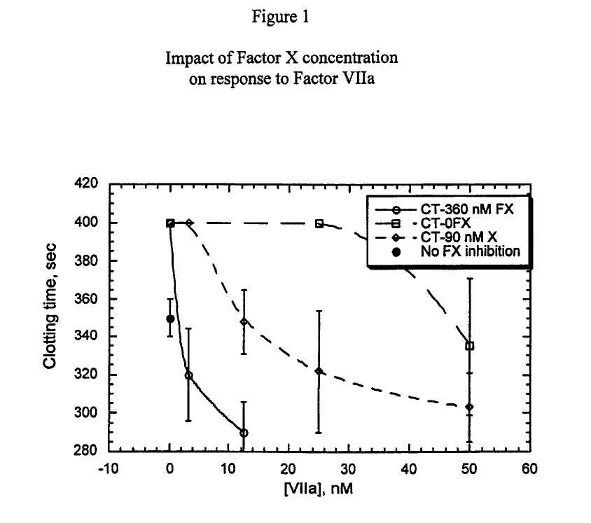

Synergy of Factor X and Factor VIIa: Factor X was added to factor VIII-deficient blood at 0, 90, and 360 nM levels, then incubated for 1 hour at room temperature. Normal factor X levels in whole blood are about 60 nM (100 nM in plasma). Factor VIIa (0 to 50 nM) was added and clotting times were recorded (as described above in Example 1). FIG. 1 provides the clotting time of each of the samples. After 60 minutes, all samples gave clotting times of >400 seconds. Adding factor X to factor VIII-deficient blood greatly increased sensitivity to factor VIIa. Addition of prothrombin at 2 times the normal prothrombin levels (1.4 nM) in blood had minimal impact on clotting time. The solid symbol shows the result when factor X was added and the blood was assayed immediately. The results show that 3 nM factor VIIa per mL blood (0.06 U/mL) produces a high response if factor X levels are sufficient.

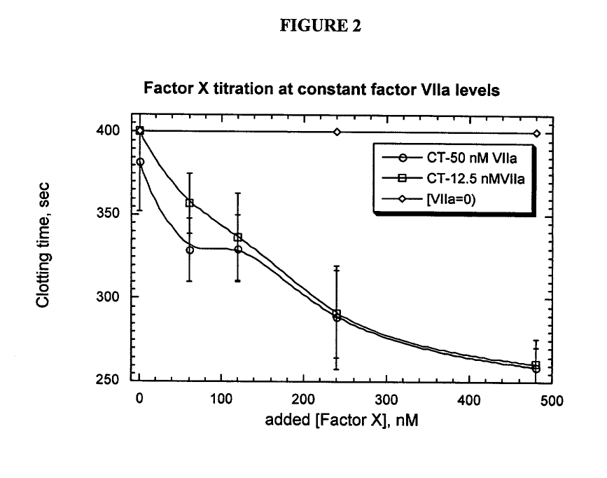

In another experiment, clotting was assessed using different concentrations of added factor X (0 to 500 nM) and a constant concentration of factor VIIa (0, 12.5 nM, or 50 nM) as described in Example 1. The results are shown in FIG. 2. The blood showed normal response to factor VIIa (380 seconds at 50 nM VIIa and >400 seconds at 12.5 nM VIIa). Clotting time decreased for samples containing factor X. At higher levels of factor X, the reaction still depended on the addition of factor VIIa, but gave the maximum response at quite low factor VIIa levels. For example, the 12.5 nM factor VIIa is well below current therapy for factor VIIa alone. In this way, the use of high factor X can provide a major sparing of factor VIIa. It should be noted that the normal blood concentration of factor X is about 60 nM, while patients on 12 hr PCC repetitive treatments have shown up to five-times the normal factor X level in their plasma. This result also shows that factor X alone is not as effective as it is in combination with factor VIIa.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a factor VIIa polypeptide and a factor X polypeptide, wherein said composition is free of thrombogenic levels of active clotting factors, other than factor VIIa, and wherein said composition contains an amount of said factor VIIa polypeptide which, upon administration to a human patient, produces an increase of 0.1 to 5 units of factor VIIa/mL of blood in said patient.

2. The composition of claim 1, wherein said composition further comprises a pharmaceutically acceptable carrier.

3. The composition of claim 1, wherein said amount of said factor X polypeptide produces, upon administration to a human patient, an increase of 1 to 10 units of factor X/mL of blood in said patient.

4. A method for increasing clot formation in a patient, said method comprising: administering to said patient an amount of a composition effective for increasing clot formation, wherein said composition comprises a factor VIIa polypeptide and a factor X polypeptide, wherein said composition is free of thrombogenic levels of active clotting factors, other than factor VIIa, and wherein said composition contains an amount of said factor VIIa polypeptide which, upon administration to a human patient, produces an increase of 0.1 to 5 units of factor VIIa/mL of blood in said patient.

5. The method of claim 4, said method further comprising monitoring clotting time of said patient.

6. A method for increasing clot formation in a patient, said method comprising:
a) providing a composition comprising a factor VIIa polypeptide and a factor X polypeptide, wherein said composition contains an amount of said factor VIIa polypeptide which, upon administration to a human patient, produces an increase of 0.1 to 5 units of factor VIIa/mL of blood in said patient;
b) inhibiting any active clotting factors other than factor VIIa within said composition to form an inhibited composition; and
c) administering to said patient an amount of said inhibited composition effective for increasing clot formation in said patient.

7. The method of claim 6, wherein said inhibiting step comprises contacting said composition with a sample of said patient's blood for a duration effective to inhibit any active clotting factors, other than factor VIIa, within said composition, wherein said contacting step is performed in the presence of a calcium chelator.

8. The method of claim 6, wherein said inhibiting step comprises contacting said composition with amounts of plasma and heparin sufficient to inhibit any active clotting factors, other than factor VIIa, within said composition, wherein said contacting step is performed in the presence of a calcium chelator.

9. The method of claim 8, wherein said heparin is low molecular weight heparin.

10. The method of claim 8, wherein said composition is contacted with an amount of heparin that results, upon administration to said patient, in less than 0.01 unit of heparin per mL of the patient's blood.

11. The method of claim 6, wherein said inhibiting step comprises contacting said composition with an amount of a protease inhibiter effective for inhibiting any active clotting factors, other than factor VIIa, within said composition.

12. The method of claim 11, wherein said protease inhibitor is antithrombin III.

13. The method of claim 11, wherein said protease inhibitor is a serine protease inhibitor.

14. The method of claim 11, wherein said contacting step further comprises contacting said composition with an amount of heparin which, upon administration to said patient, results in less than 0.01 unit per mL of said patient's blood.

15. The method of claim 6, wherein said contacting step further comprises contacting said composition with an active site inhibitor of a serine protease in the absence of calcium or in the presence of a calcium chelator.

16. The method of claim 6, said method further comprising administering a factor VIIa polypeptide to said patient.

17. The method of claim 16, said method further comprising monitoring clotting time of said patient.

18. A method for increasing clot formation in a patient, said method comprising administering to said patient a factor X polypeptide and a factor VIIa polypeptide in amounts effective for increasing clot formation in said patient, wherein the amount of said factor X polypeptide is free of thrombogenic levels of activated clotting factors, and wherein the amount of said factor VIIa produces, upon administration to a human patient, an increase of 0.1 to 5 units of factor VIIa/mL of blood in said patient.

19. The method of claim 18, wherein said method further comprises measuring blood clotting time of said patient.

20. The method of claim 18, wherein said factor X polypeptide and said factor VIIa polypeptide are administered sequentially.

21. The method of claim 18, wherein said factor VIIa polypeptide and said factor X polypeptide are administered in plasma.

22. The method of claim 18, wherein said factor VIIa polypeptide and said factor X polypeptide are administered in whole blood.

23. A composition comprising a factor VIIa polypeptide, a factor X polypeptide, anti-thrombin III, and heparin, wherein said composition is free of thrombogenic levels of active clotting factors, other than factor VIIa.

24. A composition comprising plasma or blood, a factor VIIa polypeptide, and a factor X polypeptide, wherein said composition is free of thrombogenic levels of active clotting factors, other than factor VIIa, and wherein the amount of said factor VIIa polypeptide produces, upon administration to a human subject, an increase of 0.1 to 5 units of factor VIIa/mL of blood in said human subject.

25. An article of manufacture comprising a first and a second container, wherein said first container comprises a factor VIIa polypeptide, wherein the amount of said factor VIIa polypeptide produces, upon administration to a human subject, an increase of 0.1 to 5 units of factor VIIa/mL of blood of said human subject, said second container comprises a factor X polypeptide, wherein said factor X polypeptide is free of thrombogenic levels of active clotting factors, and wherein said article of manufacture further comprises a label indicating that said factor VIIa polypeptide and said factor X polypeptide can be used to increase clot formation in a mammal.
